(19) United States
(12) Patent Application Publication
(10) Pub. No.: US 2026/0202402 A1
(43) Pub. Date: Jul. 16, 2026

(54) IMMUNOCHROMATOGRAPHIC ASSAY DEVICE

(71) Applicant: FUJIFILM CORPORATION, Tokyo (JP)

(72) Inventor: Hiroyasu ISHII, Kanagawa (JP)

(21) Appl. No.: 19/446,948

(22) Filed: Jan. 13, 2026

Related U.S. Application Data
(63) Continuation of application No. PCT/JP2024/025143, filed on Jul. 11, 2024.

(30) Foreign Application Priority Data
Jul. 21, 2023 (JP) 2023-119417

(51) Int. Cl. G01N 33/543

(52) U.S. Cl. CPC G01N 33/54388

(57) ABSTRACT

An immunochromatographic assay device includes an assay strip including an assay region and a label pad; a case accommodating the assay strip, the case including a cover member including a dropping port for adding a sample solution onto the label pad, and a case main body including a recessed portion and a support table supporting the assay strip above the inner bottom surface; a first absorbent body disposed on a side of the assay strip in a width direction, in contact with a side surface of the label pad and extending toward an inner bottom surface, the first absorbent body absorbing the sample solution by a capillary force; and a second absorbent body disposed on the inner bottom surface at a position separated from the assay strip and the first absorbent body, the second absorbent body absorbing sample solution leaked from the first absorbent body.

### CROSS-REFERENCE TO RELATED APPLICATIONS

This application is a continuation of International Application No. PCT/JP2024/025143, filed on Jul. 11, 2024, which claims priority from Japanese Patent Application No. 2023-119417, filed on Jul. 21, 2023. The entire disclosure of each of the above applications is incorporated herein by reference.

### BACKGROUND

### 1. Technical Field

The present disclosure relates to an immunochromatographic assay device.

### 2. Related Art

An immunological measuring method is widely used as a method of qualitatively or quantitatively measuring a test substance present in a biological specimen such as urine or blood. Among the above, an immunochromatographic method is highly convenient since the operation is easy and the measurement can be carried out in a short time.

In the immunochromatographic method, an assay strip, on which an antibody (a capture antibody) that specifically binds to a test substance (for example, an antigen) is immobilized in an assay region, is used. As an assay method used in the immunochromatographic method, an antibody subjected to labeling (hereinafter, referred to as a labeled antibody), which specifically binds to an antigen, and a sample are developed on an assay strip to form a complex of the capture antibody-antigen-labeled antibody in the assay region in a case where the antigen is contained in the sample. Then, a signal such as color development due to the labeling of the labeled antibody is detected to qualitatively or quantitatively measure the antigen which is a test substance.

The assay strip is generally accommodated in a case made of plastic and provided as an immunochromatographic assay device (hereinafter, simply referred to as an assay device). The assay device includes a dropping port for dropping a sample solution onto the assay strip, and a detection window for detecting a signal such as color development due to the label. In general, for a dropping amount of the sample solution, there is an appropriate amount for each assay device, and the dropping amount is described in an instruction manual or the like. However, in a case where the operator adds the sample solution dropwise, an excessive amount may be added dropwise. The assay strip includes, at a sample solution dropping position, a label pad to which the labeled antibody is applied, and the sample solution added dropwise onto the label pad dissolves the labeled antibody and flows toward the assay region in the assay strip by a capillary action. However, in a case where an excessive amount of the sample solution is added dropwise, a part of the sample solution may flow on a surface of the assay strip without permeating the label pad. In such a case, there is a possibility that determination failure such as a decrease in sensitivity or a false negative occurs.

JP2016-099283A discloses an assay device in which an absorption part that absorbs a sample solution by a capillary action is provided in proximity to a sample dropping part is provided, an excess portion of the sample solution that is excessively added dropwise is held by the absorbent part, and an appropriate amount flows on the assay strip.

JP2019-109207A and JP2019-109212A propose an assay device including a water-absorbing structure for absorbing a sample solution that is excessively added dropwise and leaked into a device.

### SUMMARY

In JP2016-099283A, as the absorbent part, a slit group that is integrally formed with the case is provided on an inner surface of the case accommodating the assay strip. In a case where the sufficiently excessive sample solution is added dropwise, it is considered that the excess sample solution can be drawn into a gap between the slits by a capillary force of the slit group. On the other hand, in a case formed of a general resin molded product, a spacing of the slits is much larger than a pore diameter of the label pad. Therefore, the capillary force of the slit group is lower than the capillary force of the label pad, and in a case where the amount of the excess sample solution is small, the sample solution cannot be drawn into the gap of the slit against the capillary force of the label pad, and the excess sample solution may not be suppressed from flowing on the surface of the assay strip. In addition, even in a case where the sample solution can be drawn into the gap of the slit, since the capillary force of the label pad is strong, the sample solution once drawn into the slit may return to the label pad side and the excessive sample solution may be developed on the assay strip.

The water-absorbing structure in JP2019-109207A and JP2019-109212A is for absorbing the excess sample solution that leaks from the sample pad and flows on an inner bottom surface of a case that accommodates the assay strip. In JP2019-109207A and JP2019-109212A, it is not considered to suppress the sample solution from flowing on the surface of the assay strip toward the assay region.

In any case, in a case where an excessive amount of the sample solution is added dropwise, measures to make the amount of the sample solution that flows on the surface of the assay strip and flows toward the assay region or the liquid amount of the sample solution supplied to the assay strip an appropriate amount are insufficient, and the occurrence of the determination failure is not sufficiently suppressed.

An object of the present disclosure is to provide an immunochromatographic assay device that can obtain a determination result with higher reliability than that of the related art even in a case where an excessive amount of the sample solution is added dropwise.

An immunochromatographic assay device according to the present disclosure includes
an assay strip on which a sample solution is developed, the strip including an assay region configured to capture a test substance contained in the sample solution, and a label pad on an upstream side of the assay region, the label pad containing a labeling substance that labels the test substance;
a case that accommodates the assay strip and includes a cover member including a dropping port for adding the sample solution dropwise onto the label pad of the assay strip, and a case main body being configured to accommodate the assay strip and including a recessed portion and a support table on an inner bottom surface of the recessed portion, the support table being configured to support the assay strip above the inner bottom surface;
a first absorbent body that is disposed, on a side of the assay strip in a width direction, in contact with at least a side surface of the label pad and in an orientation extending toward an inner bottom surface side of the case main body, the first absorbent body being configured to absorb the sample solution by a capillary force; and
a second absorbent body that is disposed, on the inner bottom surface of the case main body, at a position separated from the assay strip and the first absorber, the second absorbent body being configured to absorb the sample solution that has leaked from the first absorbent body.

In the immunochromatographic assay device, it is preferable that a liquid-holding capacity of the first absorbent body is equal to or less than a specified amount to be added dropwise onto the assay strip.

In the immunochromatographic assay device, it is preferable that a total liquid-holding capacity of the label pad and the first absorbent body is 1 to 2 times a specified amount to be added dropwise onto the assay strip.

In the immunochromatographic assay device, it is preferable that the first absorbent body consists of the same material as a material of the label pad.

In the immunochromatographic assay device, it is preferable that a contact portion between the first absorbent body and the assay strip is only the label pad.

In the immunochromatographic assay device, the first absorbent body may include a labeling substance.

In the immunochromatographic assay device, the second absorbent body may be composed of a plurality of absorbent bodies disposed around the first absorbent body.

In the immunochromatographic assay device, it is preferable that the second absorbent body has a length equal to or longer than a length of the first absorbent body along a longitudinal direction of the assay strip, and is disposed with a lengthwise direction of the second absorbent body along the longitudinal direction and facing to the first absorbent body.

In the immunochromatographic assay device, the first absorbent body may be in contact with the inner bottom surface of the case main body.

In the immunochromatographic assay device, the first absorbent body may be disposed to be separated from the inner bottom surface of the case main body, and the second absorbent body may be disposed directly below the first absorbent body.

In the immunochromatographic assay device, it is preferable that the second absorbent body has a shape surrounding the first absorbent body.

In the immunochromatographic assay device, it is preferable that the second absorbent body is disposed at a position at a distance of 1 mm or less from the first absorbent body.

In the immunochromatographic assay device, it is preferable that the first absorbent body and the second absorbent body are disposed on both sides of the assay strip, with the assay strip interposed therebetween.

According to the immunochromatographic assay device of the present disclosure, a determination result with higher reliability than that of the related art can be obtained even in a case where an excessive amount of the sample solution is added dropwise.

### BRIEF DESCRIPTION OF THE DRAWINGS

FIG. 1 is a perspective view of an assay device.

FIG. 2 is an exploded perspective view of the assay device.

FIG. 3 is a broken-away side view of the assay device.

FIG. 4 is a broken-away side view of the assay device showing a state in which a first pressing operation part is pushed.

FIG. 5 is a broken-away side view of the assay device showing a state in which a second pressing operation part is pushed in addition to the first pressing operation part.

FIG. 6 is a diagram showing a procedure of an immunochromatographic assay using the assay device.

FIG. 7 is a perspective view showing a positional relationship between an assay strip, a first absorbent body, and a second absorbent body in the assay device.

FIG. 8A is an enlarged plan view of an edge part of a label pad of the assay device shown in FIG. 7, and FIG. 8B is a broken-away side view taken along line b-b.

FIG. 9 is a cross-sectional view taken along line IX-IX in FIG. 8A.

FIG. 10 is an explanatory diagram of a contact portion between the first absorbent body and the assay strip.

FIG. 11 is a diagram showing a modification example of the first absorbent body and the second absorbent body, and FIG. 11A is an enlarged plan view and FIG. 11B is a broken-away side view taken along line b-b of an edge part of a label pad of the assay device.

FIG. 12 is a diagram showing a modification example of the first absorbent body and the second absorbent body, and FIG. 12A is an enlarged plan view and FIG. 12B is a broken-away side view taken along line b-b of an edge part of a label pad of the assay device.

FIG. 13 is a diagram showing a modification example of the first absorbent body and the second absorbent body, and FIG. 13A is an enlarged plan view and FIG. 13B is a broken-away side view taken along line b-b of an edge part of a label pad of the assay device.

FIG. 14 is a diagram showing a modification example of the first absorbent body and the second absorbent body, and FIG. 14A is an enlarged plan view and FIG. 14B is a broken-away side view taken along line b-b of an edge part of a label pad of the assay device.

FIG. 15 is a diagram showing a modification example of the first absorbent body and the second absorbent body, and FIG. 15A is an enlarged plan view and FIG. 15B is a broken-away side view taken along line b-b of an edge part of a label pad of the assay device.

### DESCRIPTION OF EMBODIMENTS

Hereinafter, an immunochromatographic assay device according to an embodiment of the present disclosure (hereinafter, referred to as an assay device) will be described with reference to the drawings. The constituent elements indicated by the same reference numerals in the drawings mean the same constituent elements.

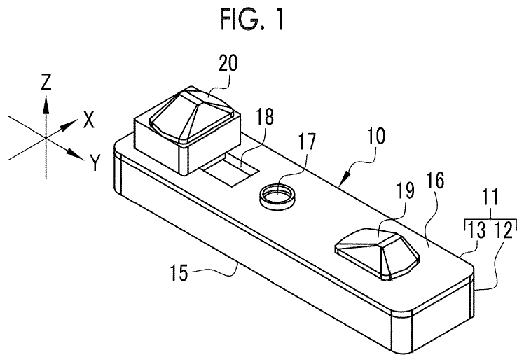

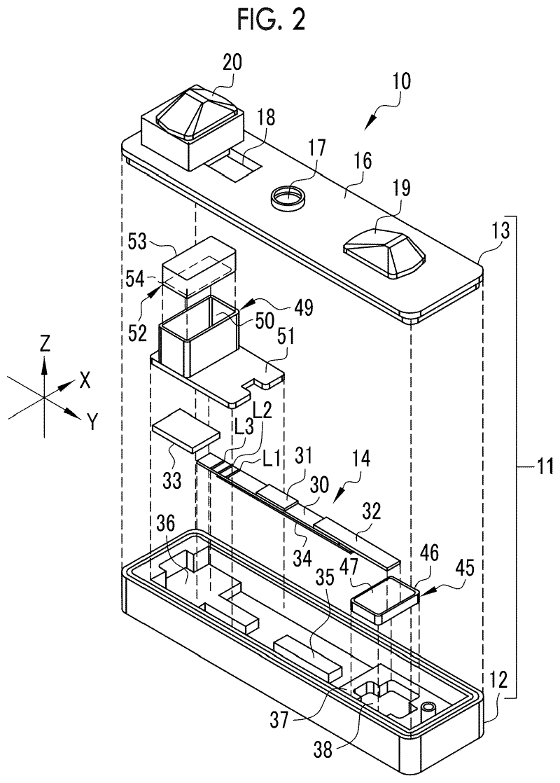

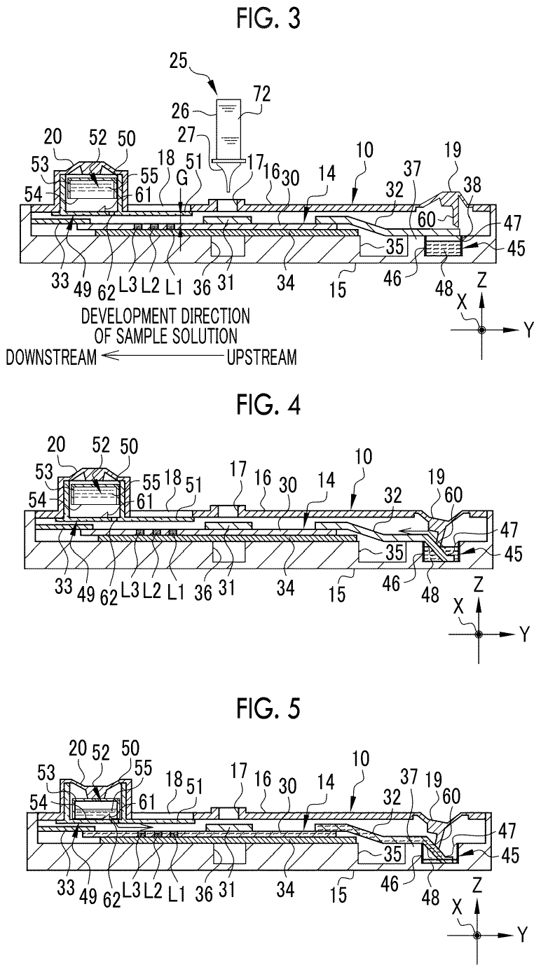

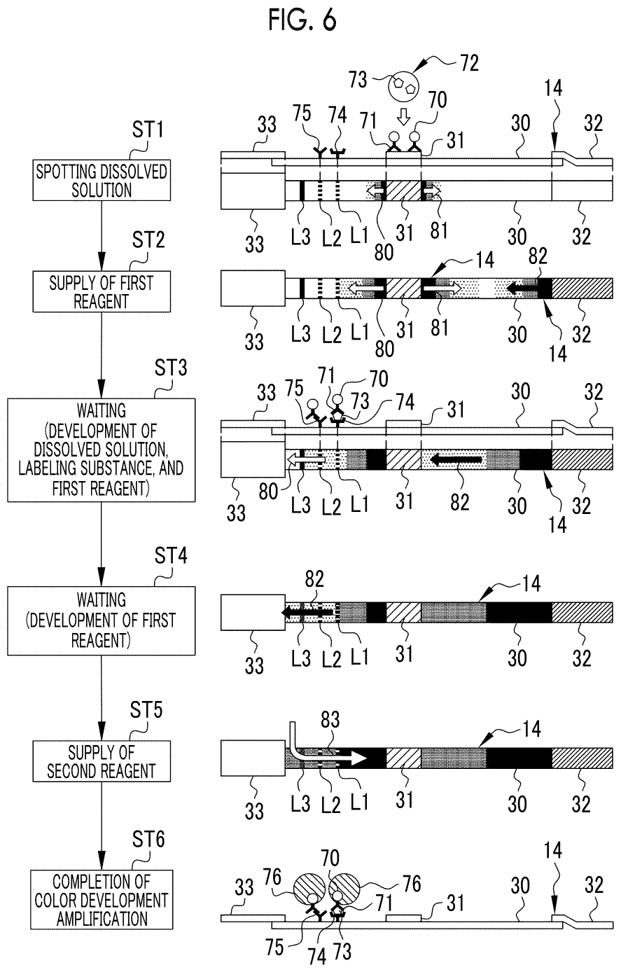

FIG. 1 is an external view of an assay device 10 according to an embodiment, and FIG. 2 is an exploded perspective view of the assay device 10. FIG. 3 is a broken-away side view of the assay device 10, FIG. 4 is a broken-away side view of the assay device 10 showing a state in which a first pressing operation part 19 is pushed, and FIG. 5 is a broken-away side view of the assay device 10 showing a state in which a second pressing operation part 20 is pushed in addition to the first pressing operation part 19. In addition, FIG. 6 is a diagram showing a procedure of an immunochromatographic assay using the assay device 10.

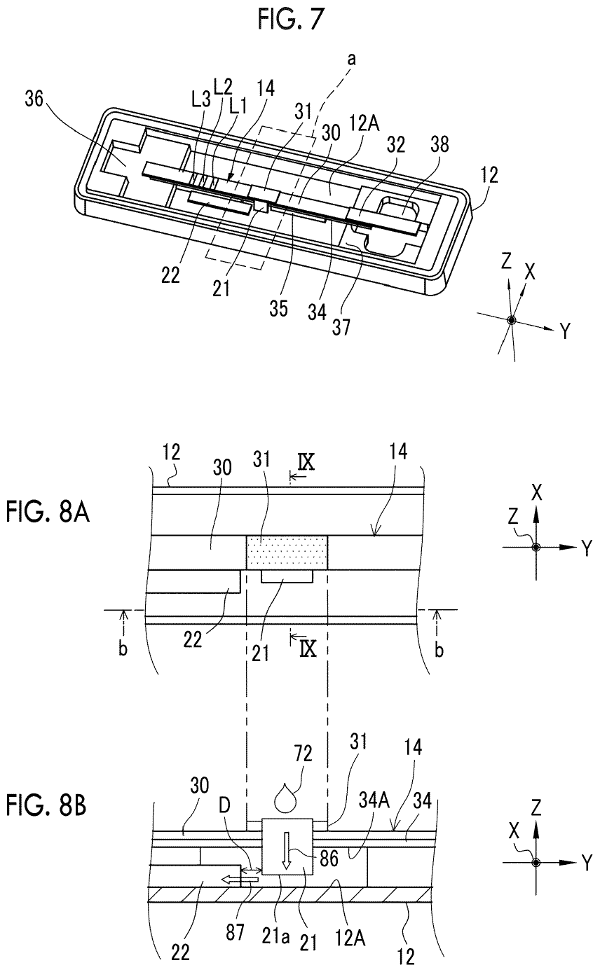

The assay device 10 is a single-use type that is used one by one in each sample of assay target. As shown in FIGS. 1 and 2, the assay device 10 includes an assay strip 14 and a case 11 that accommodates the assay strip 14. In addition, in the case 11, a first absorbent body 21, a second absorbent body 22 (see FIG. 7), a first reagent holding part 45, a second reagent holding part 52, and the like are accommodated, in addition to the assay strip 14.

The assay strip 14 has an assay region L1 in which a sample solution 72 (see FIG. 6) is developed and a test substance 73 (see FIG. 6) contained in the sample solution 72 is captured. In the assay region L1, the color development state changes depending on whether or not the sample contains a test substance 73, that is, whether the sample is positive or negative. The assay device 10 has a configuration in which the user can confirm whether the sample is positive or negative by visually observing the change in the color development state.

The assay device 10 of the present example is characterized that the first absorbent body 21 and the second absorbent body 22 are provided, but the detail of the first absorbent body 21 and the second absorbent body 22 is described after a basic configuration, a basic method for using, and the like of the assay device 10 is described.

The case 11 has an elongate rectangular parallelepiped shape and is composed of a case main body 12 and a cover member 13. The case main body 12 is a box having a recessed portion the constitutes an accommodation space that is surrounded by a bottom plate having an elongated rectangular plate shape and four side plates standing vertically from four sides of the bottom plate. An inner bottom surface 12A (see FIG. 7) of the accommodation space of the case main body 12 is an “inner bottom surface of a case main body” according to the technology of the present disclosure.

A protruding portion-shaped support bases 35 to 37 on which the assay strip 14 is placed are formed on the inner bottom surface 12A of the case main body 12. The support table 35 is provided in a central part of the case main body 12. The support table 36 is provided in one end part of the case main body 12 on the downstream side in the development direction of the sample solution 72. The support tables 35 and 36 are at the same height. The support table 37 is provided in the other end part of the case main body 12 on the upstream side in the development direction of the sample solution 72. The support table 37 is at the same height as the support tables 35 and 36. The assay strip 14 is placed on the support bases 35 to 37. The assay strip 14 is supported above the inner bottom surface 12A by the support bases 35 to 37.

The cover member 13 has an elongated rectangular plate shape similarly to the bottom plate of the case main body 12 and functions as a lid that covers the accommodation space of the case main body 12. A surface of the cover member 13 is a surface 16 of the case 11. It is noted that both the X direction and the Y direction in the drawing are directions along the horizontal plane and are orthogonal to each other. The X direction is a direction along a short side of the case 11, and is a so-called right-left direction. The Y direction is a direction along a long side of the case 11, and is a so-called front-rear direction. The Z direction is a direction along the vertical direction and is orthogonal to the X direction and the Y direction. The Z direction is a so-called up-down direction. The directions indicated by the arrows X, Y, and Z in respective figures coincide with each other.

A dropping port 17 and an observation window 18 are formed in the cover member 13. In addition, a first pressing operation part 19 and a second pressing operation part 20 are provided in the cover member 13. The dropping port 17, the observation window 18, the first pressing operation part 19, and the second pressing operation part 20 are integrally formed.

The dropping port 17 is a round hole to which a sample solution 72 (see FIG. 6) containing a sample is added dropwise, and has a boss shape in which an edge protrudes from the surface 16. The dropping port 17 is formed in a central part of the cover member 13. The dropping port 17 is an opening for adding dropwise a sample solution 72 into the inside of the case 11. In the assay strip 14 in the case 11, a label pad 31 is disposed at a position facing the dropping port 17, and the sample solution 72 added dropwise from the dropping port 17 is spotted on the label pad 31. It is noted that the sample may be any sample as long as it can contain a test substance 73 (see FIG. 6), and it is not particularly limited. The sample includes a biological specimen of an individual as a target of the immunochromatography assay, particularly an animal as well as a human, for example, blood, serum, blood plasma, spinal fluid, tear fluid, sweat, urine, feces, pus, nasal discharge, nasal swab, pharynx swab, nasal aspirate, or sputum, as well as an organ, a tissue, a mucous membrane, and skin, or swabs containing these, or an animal or plant itself or a dried form thereof. Examples of the test substance 73 include an antigen, an antibody, a protein, and a low-molecular-weight compound.

It is noted that, as shown in FIG. 3, the sample solution 72 is accommodated in a dropping tool 25 that can accommodate equal to or more than a specified amount to be added dropwise onto the assay strip 14, and is added dropwise from the dropping port 17 using the dropping tool 25. The “specified amount to be added dropwise onto the assay strip 14” is an appropriate sample solution amount predetermined for each assay device 10. The liquid amount of the sample solution 72 that is optimal for the assay is determined as the specified amount for each assay device 10. The assay device 10 is configured to obtain an accurate assay result by performing the assay with the specified amount. In general, in a case of a commercially available assay device, the specified amount is described in an instruction manual or the like thereof. The specified amount may be described as a specific numerical value, but for example, the dropping tool 25 described later is provided as an accessory together with the assay device, and for example, “one drop is added dropwise from the dropping port 17 by the dropping tool 25” is described. In such a case, one drop in a case of being added dropwise by the dropping tool 25 as the accessory is the specified amount.

The dropping tool 25 is composed of, as an example, a container 26 that accommodates a sample treatment liquid such as an extraction solution, and a nozzle 27 that is configured to engage an opening of the container 26. For example, a swab that has collected the sample is immersed in the sample treatment liquid in the container 26 to extract the sample. In this example, the sample solution 72 is a dissolved solution in which the sample is dissolved in the sample treatment liquid.

The observation window 18 is a rectangular opening for observing, from the outside, the assay region L1 or the like of the assay strip 14. The observation window 18 is formed between the dropping port 17 and the second pressing operation part 20.

The first pressing operation part 19 is provided in one end part of the cover member 13 in the Y direction. The first pressing operation part 19 is subjected to a pressing operation by a user in a case where the first reagent 48 (see FIG. 3) is supplied to the assay strip 14. The second pressing operation part 20 is provided in the other end part of the cover member 13 in the Y direction, which is on a side opposite to the first pressing operation part 19. The second pressing operation part 20 is subjected to a pressing operation by a user in a case where the second reagent 55 (see FIG. 3) is supplied to the assay strip 14.

The assay strip 14 has an elongated rectangular plate shape as a whole and has a carrier 30, a label pad 31, a liquid feeding pad 32, an absorption pad 33, and a back pressure-sensitive adhesive sheet 34.

The carrier 30 is formed of, for example, a porous insoluble material such as a nitrocellulose membrane. The label pad 31 is attached to a position of the carrier 30 facing the dropping port 17. As described above, the sample solution 72 added dropwise into the dropping port 17 is spotted on the label pad 31. That is, the label pad 31 functions as a spotting region of the sample solution 72.

The sample solution 72 spotted on the label pad 31 permeates the carrier 30 and is developed toward the one end side of the carrier 30 in the Y direction by the capillary action. An assay region L1, a control region L2, and a color development region L3 are provided on the one end side of the carrier 30 in the Y direction on which the sample solution 72 is developed. The assay region L1, the control region L2, and the color development region L3 are strip-shaped regions extending from one end to the other end of the carrier 30 in the X direction. In a case where a direction from the label pad 31 toward the assay region L1 and the like is defined as the development direction of the sample solution 72 (see FIG. 3), the assay region L1 is positioned on the most upstream side in the development direction and the color development region L3 is positioned on the most downstream side in the development direction. The control region L2 is positioned between the assay region L1 and the color development region L3. In other words, the control region L2 is positioned on the downstream side of the assay region L1 and on the upstream side of the color development region L3. In FIG. 2, the assay region L1, the control region L2, and the color development region L3 are hatched, but the hatching is for convenience of description and does not indicate that each of the regions L1 to L3 develops color. The same applies to subsequent FIGS. 3 to 5 and the like.

The liquid feeding pad 32 is attached to one end of the carrier 30, which is on a side opposite to the other end of the carrier 30 having the assay region L1 or the like. The liquid feeding pad 32 is formed from a porous material similarly to the carrier 30 and the like, and it feeds the first reagent 48 to the carrier 30 by the capillary action.

The absorption pad 33 is attached to one end of the carrier 30 on which the assay region L1 and the like are provided. The absorption pad 33 is also formed of a porous material. The absorption pad 33 absorbs the sample solution 72, the first reagent 48, and the second reagent 55 developed on the carrier 30. In this way, by actively absorbing the sample solution 72 and the like by the absorption pad 33, the development speed of the sample solution 72 and the like is increased.

The back pressure-sensitive adhesive sheet 34 is a base material of which the surface is a pressure-sensitive adhesive surface, and the carrier 30 is adhesively fixed thereto. The back pressure-sensitive adhesive sheet 34 and then the assay strip 14 are placed on protruding support tables 35 to 37 which are formed in the accommodation space of the case main body 12.

A recessed first housing part 38 is formed on the support base 37 formed in the accommodation space of the case main body 12. The first housing part 38 is provided at a position facing the other end of the liquid feeding pad 32 attached to the carrier 30, which is opposite to one end attached to the carrier 30. The first reagent holding part 45 is accommodated in the first housing part 38.

The first reagent holding part 45 is composed of a container 46 that has an opening on one surface and a seal 47 that liquid-tightly covers the opening of the container 46. The container 46 is formed from, for example, a resin material. The first reagent 48 is stored in the inside of the container 46. The seal 47 is, for example, an aluminum sheet, and it can be easily broken by a sharp blade or the like. The first reagent holding part 45 is accommodated in the first housing part 38 with the seal 47 being faced upward so that the seal 47 faces the other end of the liquid feeding pad 32.

A multifunctional member 49 is disposed in the upper part of the support table 36. The multifunctional member 49 is formed from a transparent resin material, for example, an acrylic resin. The multifunctional member 49 is a member in which a second housing part 50 and a flow channel forming part 51 are integrally provided. The second housing part 50 is a box of which the upper surface is open, and the second reagent holding part 52 is accommodated in the inside of the second housing part 50. The flow channel forming part 51 is a flat plate that extends from the bottom part of the second housing part 50 in the Y direction. The flow channel forming part 51 extends to the front of the label pad 31 and covers the upper part of the assay region L1, the control region L2, and the color development region L3 of the carrier 30. A spacing G is provided between the carrier 30 and the flow channel forming part 51 (see FIG. 3). The spacing G is, for example, in a range of 0.01 mm to 1 mm. Since the spacing G is provided between the carrier 30 and the flow channel forming part 51 in this way, a flow channel of the second reagent 55 is ensured.

The second reagent holding part 52 is composed of a container 53 that has an opening on one surface and a seal 54 that liquid-tightly covers the opening of the container 53. The container 53 is formed from, for example, a resin material. The second reagent 55 is stored in the inside of the container 53. The seal 54 is, for example, an aluminum sheet, and it can be easily broken by a sharp blade or the like. The second reagent holding part 52 is accommodated in the second housing part 50 with the seal 54 being faced downward so that the seal 54 faces the assay strip 14.

As shown in FIG. 3 as an example, a first breaking protrusion 60 is provided in the first pressing operation part 19. The second reagent holding part 52 is attached to an inner surface of the second pressing operating part 20. A second breaking protrusion 61 and a supply port 62 are provided at the bottom part of the second housing part 50. The distal end of each of first breaking protrusion 60 and the second breaking protrusion 61 is sharp.

As shown in FIG. 4 as an example, in a case where the first pressing operation part 19 is subjected to a pressing operation, the first breaking protrusion 60 abuts on the other end of the liquid feeding pad 32 and pushes the other end of the liquid feeding pad 32 toward the seal 47 of the first reagent holding part 45. The seal 47 is broken due to the pushing of the other end of the liquid feeding pad 32 by the first breaking protrusion 60, and the other end of the liquid feeding pad 32 falls into the container 46 to be immersed in the first reagent 48. The first reagent 48 is developed from the other end of the liquid feeding pad 32 toward the carrier 30 by the capillary action. The first pressing operation part 19 is maintained in a crushed state even after being crushed by the pressing operation. Therefore, the spreading of the first reagent 48 is continued until substantially the entire first reagent 48 is sucked up to the liquid feeding pad 32.

As shown in FIG. 5 as an example, in a case where the second pressing operation part 20 is subjected to a pressing operation, the second reagent holding part 52 is moved downward in the second housing part 50 and reaches the bottom part of the second housing part 50 having the second breaking protrusion 61. Then, the seal 54 of the second reagent holding part 52 is broken by the second breaking protrusion 61. The second reagent 55 stored in the second reagent holding part 52 flows from the broken portion of the seal 54 to the supply port 62. Further, it flows through the flow channel ensured by the spacing G (see FIG. 3) and then is supplied to the carrier 30.

As shown in FIG. 6 as an example, the label pad 31 includes a labeling substance 70. The labeling substance 70 is modified with a first binding substance 71. The first binding substance 71 specifically binds to the test substance 73 contained in the sample solution 72. In the present embodiment, for example, gold colloid particles having a diameter of 50 nm (manufactured by BBI Solutions, product code: EM.GC50) are used as the labeling substance 70. It is noted that the labeling substance 70 is not limited to the gold colloid particles, and may be a metal sulfide used in an immunochromatographic method, coloring particles used in an immunoagglutination reaction, or the like, and a metal colloid is preferable. Examples of the metal colloid include a silver colloid, a platinum colloid, an iron colloid, an aluminum hydroxide colloid, and a composite colloid thereof, in addition to the above-described gold colloid. In particular, at an appropriate particle diameter, a gold colloid is preferable since it exhibits a red color, and a silver colloid is preferable since it exhibits a yellow color, among which a gold colloid is most preferable.

For example, in a case where the test substance 73 is an antigen, the first binding substance 71 is an antibody against the antigen, and in a case where the test substance 73 is an antibody, the first binding substance 71 is an antigen against the antibody. In a case where the test substance 73 is a protein, a low-molecular-weight compound, or the like, the first binding substance 71 is an aptamer with respect to the protein, the low-molecular-weight compound, or the like.

The assay region L1 includes a second binding substance 74. The second binding substance 74 specifically binds to the test substance 73 to which the first binding substance 71 is bound. As a result, the test substance 73 is captured in the assay region L1. In a case where the test substance 73 is captured, the color optical density of the assay region L1 increases to be equal to or more than a preset criterion. In a case where the color optical density of the assay region L1 increases to be equal to or more than the criterion, it is found that the test substance 73 is contained in the sample, that is, the sample is positive.

Before the sample solution 72 is developed, the assay region L1 is substantially the same color (for example, white) as the carrier 30. The assay region L1 appears as a line in a case where the sample solution 72 is developed and the test substance 73 is contained in the developed sample solution 72, that is, in a case where the sample is positive, by increasing the color optical density. The assay region L1 is colored black because it is amplified by silver amplification described later.

It is noted that the change in the color development state of the assay region L1 includes any of discoloration, color development, or color density change. The discoloration is an aspect in which the color of the carrier 30 changes from a first color to a second color different from the first color. The color development is an aspect in which a color different from the color of the carrier 30 is developed, and the color of the carrier 30 changes to the different color. The color density change is an aspect in which the density of the color changes.

For example, similarly to the first binding substance 71, in a case where the test substance 73 is an antigen, the second binding substance 74 is an antibody against the antigen, and in a case where the test substance 73 is an antibody, the second binding substance 74 is an antigen against the antibody. In a case where the test substance 73 is a protein, a low-molecular-weight compound, or the like, the second binding substance 74 is an aptamer with respect to the protein, the low-molecular-weight compound, or the like. The first binding substance 71 and the second binding substance 74 may be the same as or different from each other. For example, in a case where the test substance 73 is an influenza A type virus or a biomarker thereof, it is possible to use an anti-influenza A type monoclonal antibody (manufactured by Medix Biochemica Inc., product name: Anti-influenza A SPT N-5 7307) as the first binding substance 71 and the second binding substance 74.

The control region L2 includes a third binding substance 75. The third binding substance 75 specifically binds to the first binding substance 71. As a result, the labeling substance 70 is captured in the control region L2. Among the labeling substances 70, the first binding substance 73 may not bind to the test substance 71. Such a labeling substance 70 reaches the control region L2 without being captured in the assay region L1 and then is captured in the control region L2. In a case where the labeling substance 70 is captured, the color optical density of the control region L2 increases to be equal to or more than a preset criterion. In a case where the color optical density of the control region L2 increases to be equal to or more than the criterion, it is found that the sample solution 72 is sufficiently developed on the carrier 30 and the development of the sample solution 72 is completed.

The control region L2 is also substantially the same color (for example, white) as the carrier 30 before the sample solution 72 is developed, similarly to the assay region L1. The control region L2 appears as a line in a case where the color optical density increases in a case where the labeling substance 70 is captured. The control region L2 is also colored black because it is amplified by silver amplification described later.

The third binding substance 75 may be the test substance 73 itself or a compound having a site recognized by the first binding substance 71. Examples of the compound having a moiety that is recognized by the first binding substance 71 include such a compound that is obtained by bonding a derivative of the test substance 73 to a protein. For example, in a case where the test substance 73 is an influenza A type virus or a biomarker thereof, it is possible to use, as the third binding substance 75, an anti-mouse IgG antibody (manufactured by Fujifilm Wako Pure Chemical Corporation, product name: anti-mouse IgG (H+L), rabbit F(ab′)2, product code: 566-70621).

The color development region L3 contains a substance (not shown) of which the color development state changes by reacting with the first reagent 48. In a case where the color development region L3 reacts with the first reagent 48 to develop color or the color changes, it is found that the first reagent 48 is developed to the color development region L3 and the timing of starting the supply of the second reagent 55 is reached. For example, in a case where a mixed aqueous solution of an aqueous iron nitrate solution and a citric acid (manufactured by FUJIFILM Wako Pure Chemical Corporation, product code: 038-06925) is used as the first reagent 48, it is preferable to constitute the color development region L3 with a color development reagent-immobilized line in which bromocresol green manufactured by FUJIFILM Wako Pure Chemical Corporation is immobilized in a band shape. In this case, the color development region L3 is dark green before reacting with the first reagent 48, and changes to orange in a case where the first reagent 48 reaches.

The first reagent 48 and the second reagent 55 are an amplification solution that amplifies the color development of the assay region L1 and the control region L2 by reacting with each other. In a case where a metal-based substance such as gold colloid particles is used as the labeling substance 70 as in the present example, silver amplification is used as a method of amplifying the color development. The first reagent 48 and the second reagent 55 are, as an example, an amplification solution used for silver amplification. The reaction of the first reagent 48 and the second reagent 55 using the labeling substance 70 as a catalyst is an amplification reaction. By the amplification reaction, silver particles 76 having a particle diameter relatively larger than that of the labeling substance 70 are generated.

More specifically, the first reagent 48 is a solution of a reducing agent that reduces silver ions, and the second reagent 55 is a solution of silver ions. In a case where the reducing agent in the first reagent 48 and the silver ions in the second reagent 55 are brought into contact with the labeling substance 70, the silver particles 76 are generated. The silver particles 76 are deposited on the labeling substance 70 with the labeling substance 70 as a nucleus. The silver particles 76 deposited on the labeling substance 70 have a particle diameter of several tens to several hundreds of times the labeling substance 70 in the end. Accordingly, the labeling signal issued by the labeling substance 70 is amplified, and as a result, the color development in the assay region L1 and the control region L2 is amplified.

As the reducing agent of the first reagent 48, any inorganic or organic material or a mixture thereof can be used as long as these can reduce silver ions into silver. Preferred examples of the inorganic reducing agent include a reducing metal salt and a reducing metal complex salt, of which the atomic valence is capable of being changed with a metal ion such as Fe²⁺, V²⁺, or Ti³⁺. In a case where an inorganic reducing agent is used, oxidized ions need to be removed or made harmless by the formation of a complex or reduction. For example, in a case where Fe²⁺ is used as the reducing agent, a complex of Fe³⁺, which is an oxide, is formed using citric acid or ethylenediaminetetraacetic acid (EDTA), and therefore detoxification is possible. In the present example, an inorganic reducing agent is preferably used as the reducing agent, and a metal salt of Fe²⁺ is more preferably used.

It is also possible to use, as the reducing agent, a developing agent used in a light-sensitive silver halide photographic material of a wet-type (for example, methyl gallate, hydroquinone, substituted hydroquinone, 3-pyrazolidones, p-aminophenols, p-phenylenediamines, hindered phenols, amidoximes, azines, catechols, pyrogallols, ascorbic acid (or a derivative thereof), and leuco dyes), and other materials obvious to those who are skilled in the related art in the present field, for example, a material described in U.S. Pat. No. 6,020,117A.

As the reducing agent, an ascorbic acid reducing agent is also preferable. The useful ascorbic acid reducing agent includes ascorbic acid, an analogue thereof, an isomer thereof, and a derivative thereof. Preferred examples thereof include D- or L-ascorbic acid and a sugar derivative thereof (for example, γ-lactoascorbic acid, glucoascorbic acid, fucoascorbic acid, glucoheptoascorbic acid, or maltoascorbic acid), a sodium salt of ascorbic acid, a potassium salt of ascorbic acid, isoascorbic acid (or L-erythroascorbic acid), a salt thereof (for example, an alkali metal salt, an ammonium salt, or a salt known in the related technical field), ascorbic acid of the enediol type, ascorbic acid of the enaminol type, ascorbic acid of the thioenol type. Particularly, D-ascorbic acid, or L-ascorbic acid (and an alkali metal salt thereof) or isoascorbic acid (or an alkali metal salt thereof) is preferable, and a sodium salt is a preferred salt. A mixture of these reducing agents can be used as necessary.

The second reagent 55 is preferably a solution obtained by dissolving a silver ion-containing compound in a solvent. As the silver ion-containing compound, an organic silver salt, an inorganic silver salt, or a silver complex can be used. An inorganic silver salt or a silver complex is preferable. As the inorganic silver salt, it is possible to use a silver ion-containing compound having a high solubility in solvents such as water, and examples thereof include silver nitrate, silver acetate, silver lactate, silver butyrate, and silver thiosulfate. Silver nitrate is particularly preferable. The silver complex is preferably a silver complex in which silver is coordinated with a ligand having a water-soluble group such as a hydroxyl group or a sulfone group, and examples thereof include silver hydroxythioether.

A procedure of the immunochromatographic assay using the assay device 10 will be described with reference to FIG. 6. Here, a description will be made using, as an example, a case where a sample contains the test substance 73, that is, a case where the sample is positive. It is noted that in FIG. 6, the back pressure-sensitive adhesive sheet 34 is not illustrated in the drawing.

First, as shown in Step ST1, the sample solution 72 is spotted on the label pad 31. The test substance 73 in the sample solution 72 spotted on the label pad 31 specifically binds to the first binding substance 71 that modifies the labeling substance 70 of the label pad 31. The sample solution 72 permeates from the label pad 31 to the carrier 30, and is developed on a side (downstream side) of the assay region L1 and the like by the capillary action as indicated by an arrow 80. A part of the sample solution 72 is also developed on a side (upstream side) of the liquid feeding pad 32 as indicated by an arrow 81.

Next, as shown in Step ST2, the first reagent 48 is supplied by the pressing operation of the first pressing operation part 19. As indicated by an arrow 82, the first reagent 48 is supplied from the liquid feeding pad 32 to the carrier 30 and is developed on a side (downstream side) of the assay region L1 and the like by the capillary action.

Thereafter, as shown in Step ST3, the sample solution 72, the labeling substance 70, and the first reagent 48 are developed. The sample solution 72 and the labeling substance 70 developed from the label pad 31 are developed on a side of the assay region L1 and the like to be washed away by the first reagent 48. Finally, as shown in Step ST4, the first reagent 48 reaches the color development region L3. The color development region L3 reacts with the first reagent 48 to change the color development state. In the present example, the color development region L3 is dark green before reacting with the first reagent 48, and changes to orange by reacting with the first reagent 48.

The test substance 73 that has reached the assay region L1, more specifically, the labeling substance 70 bonded to the test substance 73 via the first binding substance 71 is captured by the second binding substance 74 of the assay region L1. As a result, the color optical density of the assay region L1 increases to be equal to or more than the criterion. On the other hand, the labeling substance 70 that is not bound to the test substance 73 passes through the assay region L1 without being captured by the second binding substance 74, and is captured by the third binding substance 75 of the control region L2. As a result, the color optical density of the control region L2 increases to be equal to or more than the criterion.

The user performs the pressing operation of the first pressing operation part 19 to develop the first reagent 48, and then waits until the first reagent 48 reaches the color development region L3 and the color development state of the color development region L3 changes (Steps ST3 and ST4). In a case where the change in the color development state of the color development region L3 is visually recognized through the observation window 18, the user performs the pressing operation of the second pressing operation part 20.

As shown in Step ST5, the second reagent 55 is supplied by the pressing operation of the second pressing operation part 20. As indicated by an arrow 83, the second reagent 55 is supplied to the carrier 30 from a downstream side of the color development region L3 and is developed on a side (upstream side) of the liquid feeding pad 32. As shown in Step ST6, the silver particles 76 are generated with the labeling substance 70 captured in the assay region L1 and the control region L2 as a nucleus by the silver ions contained in the second reagent 55 and the reducing agent contained in the first reagent 48. The color development of the assay region L1 and the control region L2 is amplified by the silver particles 76. As described above, the assay device 10 has a configuration in which the user can visually recognize whether or not the sample is positive. It is noted that the present assay device 10 can also be used in an aspect in which the assay device 10 is loaded into an assay apparatus that determines whether or not the sample is positive, and the assay apparatus presents an assay result.

As described above, the assay device 10 further includes the first absorbent body 21 and the second absorbent body 22 in the case main body 12.

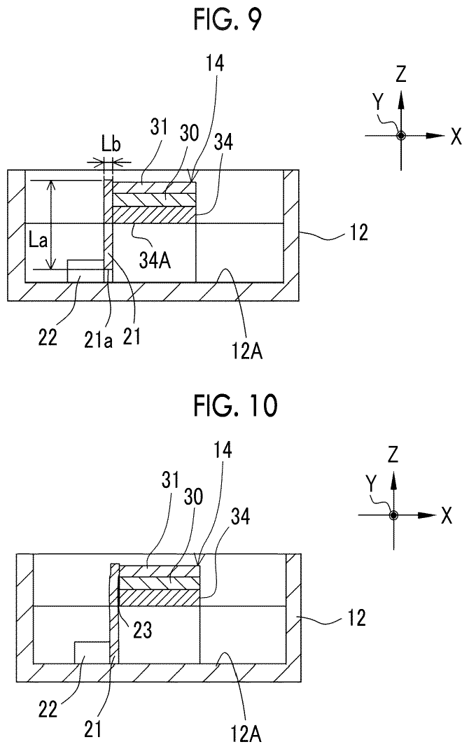

Referring to FIG. 7 and the subsequent figures, details of the disposition and functions of the first absorbent body 21 and the second absorbent body 22 in the assay device 10 will be described. FIG. 7 is a diagram showing the disposition of the first absorbent body 21 and the second absorbent body 22 in the case main body 12 of the assay device 10 shown in FIGS. 1 to 5. FIG. 8A is an enlarged plan view of a vicinity of the label pad 31 surrounded by a broken line a in FIG. 7, and FIG. 8B is a cross-sectional view taken along line b-b in FIG. 8A. FIG. 9 is a cross-sectional view taken along line IX-IX of FIG. 8A.

As shown in FIG. 7, the first absorbent body 21 is disposed on a side of the assay strip 14 in a width direction and in contact with at least a side surface of the label pad 31. In addition, as shown in FIGS. 8 and 9, the first absorbent body 21 is disposed in an orientation extending toward the inner bottom surface 12A side of the case main body 12. The disposition in an orientation extending toward the inner bottom surface 12A side means that the first absorbent body 21 is disposed in an orientation in which a lower end 21a of the first absorbent body 21 in the vertical direction is positioned on the inner bottom surface 12A side of at least a back surface of the assay strip 14, that is, a back surface 34A of the back pressure-sensitive adhesive sheet 34 as shown in FIG. 8B. In FIG. 8, the lower end 21a of the first absorbent body 21 is not in contact with the inner bottom surface 12A of the case main body 12, but the lower end 21a of the first absorbent body 21 may be in contact with the inner bottom surface 12A.

In the present example, the first absorbent body 21 has a sheet shape, and is disposed such that a thickness direction thereof matches a width direction (X direction in the figure) of the assay strip 14 as shown in FIG. 9. In the present example, the first absorbent body 21 is disposed such that a length La in the vertical direction is longer than a length (that is, the thickness of the sheet) Lb in the horizontal direction.

The first absorbent body 21 absorbs at least a part of the sample solution 72 added dropwise onto the label pad 31. The first absorbent body 21 is formed of a porous material, and absorbs the sample solution 72 from a portion in contact with the label pad 31 by a capillary force. In particular, in a case where the sample solution 72 having a liquid amount equal to or more than the specified amount is added dropwise onto the label pad 31, the excess sample solution 72 that is not completely absorbed by the label pad 31 is absorbed.

The sample solution 72 absorbed by the first absorbent body 21 moves vertically downward in the first absorbent body 21 by a capillary force and/or a self-weight as indicated by an arrow 86 in FIG. 8B. As the liquid amount of the sample solution 72 in the first absorbent body 21 increases, the sample solution 72 is transferred vertically downward in the first absorbent body 21, leaks from the lower end 21a, and spreads on the inner bottom surface 12A of the case main body 12.

The second absorbent body 22 is disposed at a position separated from the assay strip 14 and the first absorbent body 21 on the inner bottom surface 12A of the case main body 12. “The disposition at a position separated from the assay strip 14 and the first absorbent body 21” means that the second absorbent body 22 is not in contact with the assay strip 14 or the first absorbent body 21. It is noted that a distance D between the second absorbent body 22 and the first absorbent body 21 is preferably 1 mm or less. It is noted that the distance D is a shortest distance between the first absorbent body 21 and the second absorbent body 22. The second absorbent body 22 absorbs the sample solution 72 that has leaked from the first absorbent body 21, that is, the sample solution 72 that has leaked from the first absorbent body 21 and has spread on the inner bottom surface 12A. An arrow 87 in FIG. 8B indicates a state in which the sample solution 72 that has leaked to the inner bottom surface 12A is absorbed by the second absorbent body 22 and moves.

As described above, the assay device 10 according to the present embodiment includes the first absorbent body 21 that is disposed on the side of the assay strip 14 in the width direction in contact with at least the side surface of the label pad 31 and in an orientation extending toward the inner bottom surface 12A side of the case main body 12. The first absorbent body 21 absorbs the sample solution 72 by a capillary force. Since the first absorbent body 21 is in contact with the label pad 31, the sample solution 72 can be excessively added dropwise onto the label pad 31, and the sample solution 72 that is not completely absorbed by the label pad 31 can be drawn into the first absorbent body 21. Since the first absorbent body 21 has a portion disposed in an orientation extending toward the inner bottom surface 12A side of the case main body 12, that is, disposed below the back surface of the assay strip 14 in the vertical direction, the sample solution 72 drawn from the label pad 31 into the first absorbent body 21 falls downward by a self-weight to the inner bottom surface 12A side and leaks to the inner bottom surface 12A. Therefore, the capillary force for sucking the sample solution 72 of the first absorbent body 21 is always in a state of having a margin, and the sample solution 72 that is excessively added dropwise can be quickly drawn without a decrease in the capillary force in a portion in contact with the label pad 31 above the inner bottom surface 12A. As a result, the sample solution 72 can be suppressed from flowing on the assay strip 14 in a case where the sample solution 72 is excessively added dropwise.

Furthermore, the assay device 10 includes the second absorbent body 22 that is disposed at a position separated from the assay strip 14 and the first absorbent body 21 on the inner bottom surface 12A of the case main body 12, and the second absorbent body 22 absorbs the sample solution 72 that has leaked from the first absorbent body 21. As described above, the sample solution 72 that is not completely held by the first absorbent body 21 and has leaked to the inner bottom surface 12A is absorbed by the second absorbent body 22. Since the second absorbent body 22 is disposed to be separated from the first absorbent body 21 and the assay strip 14, the sample solution 72 absorbed by the second absorbent body 22 does not return to the assay strip 14. Therefore, the excessive sample solution 72 that is supplied to the assay strip 14 can be suppressed.

As described above, since the assay device 10 includes the first absorbent body 21 and the second absorbent body 22, the determination failure such as a decrease in sensitivity or a false negative can be suppressed as compared with the assay device in the related art that does not include the first absorbent body 21 and the second absorbent body 22. As a result, according to the assay device 10, a determination result with high reliability can be obtained.

In addition, since the configuration is such that the excessive sample solution 72 does not return to the assay strip 14, in the assay device 10 of the type in which a reagent such as a reducing agent is developed after a certain time after the dropwise addition of the sample solution 72 as in the present embodiment, there is also an effect of suppressing a delay in the assay time. More specific description thereof is as follows.

As described with reference to FIG. 6, in the assay device 10 according to the present embodiment, the sample solution 72 spotted on the label pad 31 is developed on a side (downstream side) of the assay region L1 and a side (upstream side) of the liquid feeding pad 32 in the assay strip 14. In a case where the dropping amount is excessive, the amount of the liquid to be developed on the upstream side also increases. The first reagent 48 is supplied by the pressing operation of the first pressing operation part 19 after the dropwise addition of the sample solution 72, but in a case where the liquid amount of the sample solution 72 to be developed on the upstream side is large, the development of the first reagent 48 does not progress, which leads to a delay in the assay time. By including the first absorbent body 21, the excessive sample solution 72 can be absorbed, so that the sample solution 72 can be suppressed from being excessively supplied to the assay strip 14. On the other hand, in a case where the excess sample solution 72 absorbed by the second absorbent body 22 is in contact with at least one of the first absorbent body 21 or the assay strip 14, the sample solution 72 in the second absorbent body 22 may be reabsorbed by the capillary force of the carrier 30 or the like of the first absorbent body 21 or the assay strip 14. In a case where the liquid amount of the sample solution 72 supplied to the assay strip 14 by the reabsorption increases, the development of the first reagent 48 is delayed, which also leads to a delay in the assay time. However, as described above, in the assay device 10 according to the present embodiment, since the second absorbent body 22 is not in contact with the first absorbent body 21 or the assay strip 14, the reabsorption of the sample solution 72 that has absorbed by the second absorbent body 22 into the assay strip 14 can be suppressed. Therefore, in the assay device 10, an effect of suppressing the delay in the assay time can be obtained.

In addition, in the assay device 10, in a case where the second absorbent body 22 is disposed at a position within 1 mm from the first absorbent body 21, the excess sample solution 72 that has leaked from the first absorbent body 21 can be quickly absorbed by the second absorbent body 22.

In the assay device 10, it is preferable that a liquid-holding capacity of the first absorbent body 21 is equal to or less than a specified amount to be added dropwise onto the assay strip 14.

Since the first absorbent body 21 is in contact with the label pad 31, in a case where the sample solution 72 in the label pad 31 flows to the downstream side of the assay strip 14, the label pad 31 sucks the sample solution 72 in the first absorbent body 21 by the capillary force and supplies the sample solution 72 to the assay strip 14. In a case where a liquid-holding capacity of the first absorbent body 21 is equal to or less than the specified amount, the excessive sample solution 72 can be more effectively suppressed from flowing to the assay strip 14. It is noted that in a case where the first absorbent body 21 is composed of a plurality of absorbent bodies, it is preferable that the total liquid-holding capacity of the plurality of absorbent bodies is equal to or less than the above-described specified amount.

It is preferable that a total liquid-holding capacity of the label pad 31 and the first absorbent body 21 is 1 to 2 times a specified amount to be added dropwise onto the assay strip 14. Similarly to the above-described case, in a case where the first absorbent body 21 is composed of a plurality of absorbent bodies, it is preferable that the liquid-holding capacity of the label pad 31 and the plurality of absorbent bodies constituting the first absorbent body 21 is 1 to 2 times the specified amount.

Since the amount of the sample solution that flows to the assay strip 14 can be reliably suppressed to 2 times or less the specified amount, a determination result with high reliability can be obtained.

It is preferable that the first absorbent body 21 exhibits a capillary force equal to or lower than that of the label pad 31. For example, in a case where the first absorbent body 21 is composed of the same material as the material of the label pad 31, the first absorbent body 21 can have a capillary force equal to the capillary force of the label pad 31.

In a case where the material of the first absorbent body 21 is the same as the material of the label pad 31, the sample solution 72 can be sucked by the capillary force equal to that of the label pad 31 at the time of dropwise addition of the sample solution 72, and in a case where the excessive sample solution 72 is added dropwise onto the label pad 31, the sample solution 72 can be efficiently sucked, and the excessive sample solution 72 can be effectively suppressed from flowing on the surface of the assay strip 14.

It is noted that in a case where the capillary force of the first absorbent body 21 is equal to or less than the capillary force of the label pad 31, the sample solution 72 held by the first absorbent body 21 is likely to return to the label pad 31 side.

In addition, the first absorbent body 21 may include the labeling substance 70 contained in the label pad 31. In a case where the first absorbent body 21 is in contact not only with the label pad 31 but also with, for example, the carrier 30, the sample solution 72 in the first absorbent body 21 may be returned to the carrier 30 from the first absorbent body 21 by the capillary force of the carrier 30 without returning to the label pad 31. In such a case, the sample solution 72 and the labeling substance 70 cannot sufficiently contact each other, and the labeling of the test substance 73 may be insufficient, but in a case where the first absorbent body 21 includes the labeling substance 70, the test substance 73 can be sufficiently brought into contact with the labeling substance 70 even in a case where the sample solution 72 is returned to the assay strip 14 without passing through the label pad 31.

As described above, the first absorbent body 21 has a function of absorbing the sample solution 72 that is excessively added dropwise onto the label pad 31 and guiding the sample solution 72 to the inner bottom surface 12A of the case main body 12. In the example shown in FIGS. 7 to 9, the first absorbent body 21 is not in contact with the inner bottom surface 12A and is supported by a support part not shown. However, as shown in FIG. 10, the first absorbent body 21 may be in contact with the inner bottom surface 12A of the case main body 12. FIG. 10 is a cross-sectional view similar to FIG. 9, and shows a modification example of the first absorbent body 21. In a case where the first absorbent body 21 is in contact with the inner bottom surface 12A of the case main body 12, the sample solution 72 absorbed by the first absorbent body 21 can be guided to the inner bottom surface 12A of the first absorbent body 21.

Furthermore, as shown in FIG. 10, it is preferable that a sealing member 23 consisting of an adhesive or the like is applied between the first absorbent body 21, and the carrier 30 and the back pressure-sensitive adhesive sheet 34 of the assay strip 14. In the example shown in FIG. 10, the contact portion between the first absorbent body 21 and the assay strip 14 is limited to only the label pad 31 by the sealing member 23. The sealing member 23 may be a member that suppresses the sample solution 72 from moving between the first absorbent body 21, and the carrier 30 and the back pressure-sensitive adhesive sheet 34. The sealing member 23 may be a member that does not allow the passage of a liquid, such as a resin film, in addition to the adhesive.

In a case where the contact portion between the first absorbent body 21 and the assay strip 14 is only the label pad 31, in a case where the sample solution 72 in the first absorbent body 21 is returned to the assay strip 14, the sample solution 72 always passes through the label pad 31, so that the sample solution 72 and the labeling substance 70 can be reliably brought into contact with each other.

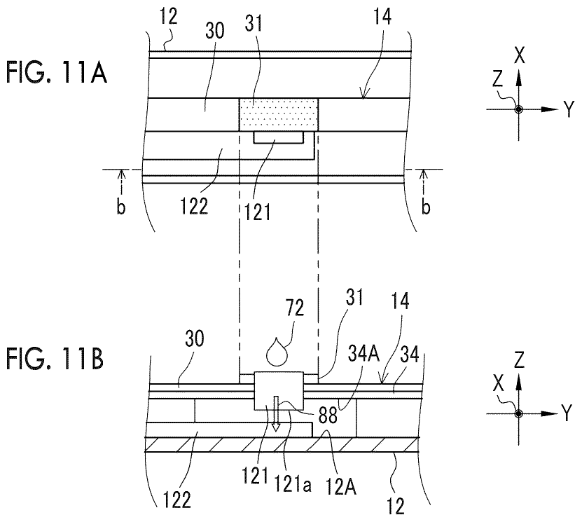

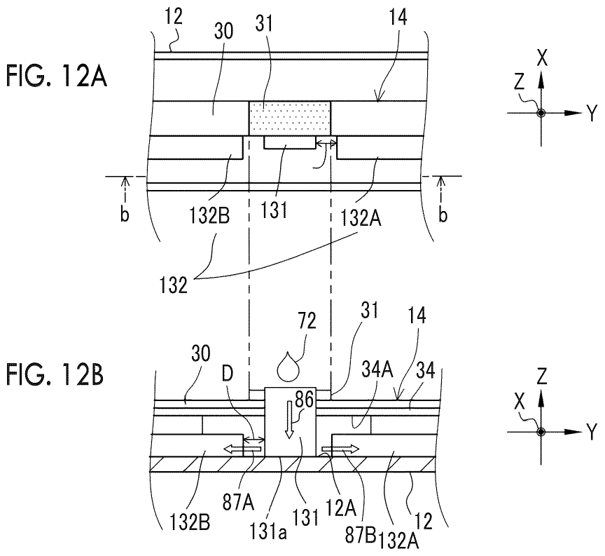

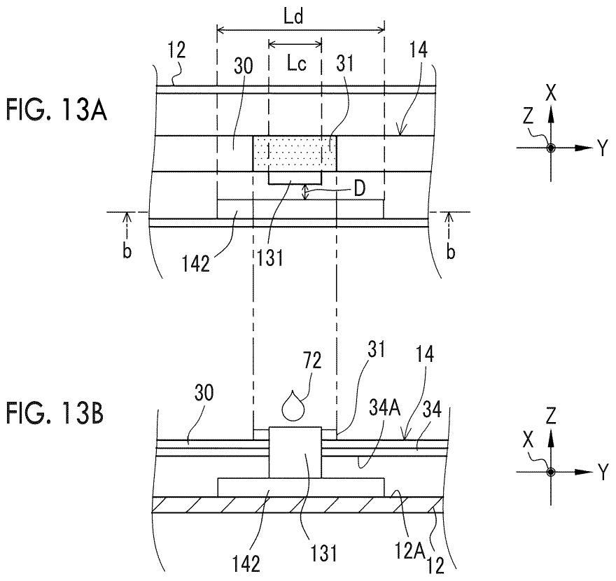

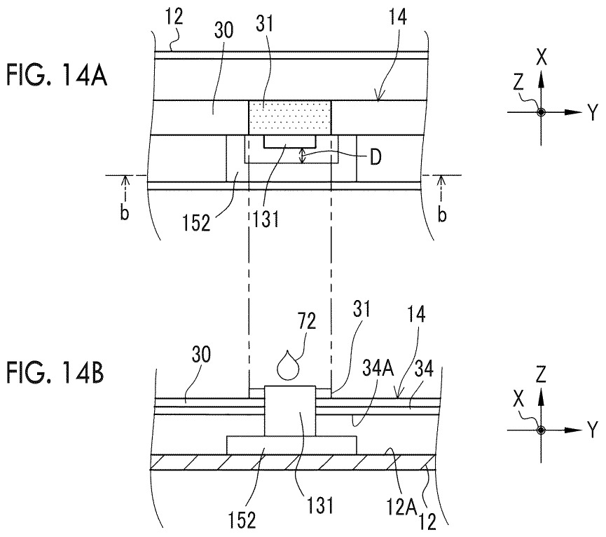

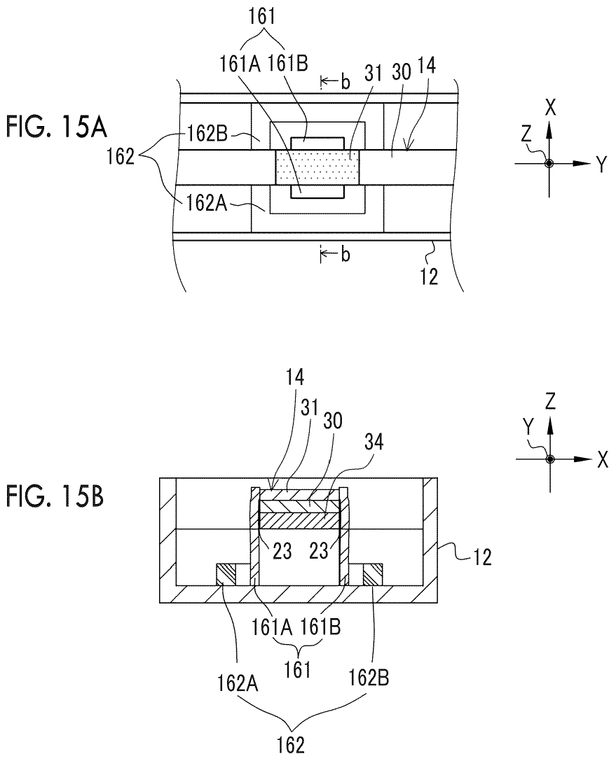

Hereinafter, modification examples of the first absorbent body and the second absorbent body will be described with reference to FIGS. 11 to 15. FIG. 11A and FIG. 11B, FIG. 12A and FIG. 12B, FIG. 13A and FIG. 13B, and FIG. 14A and FIG. 14B correspond to FIG. 8A and FIG. 8B, and are plan views and cross-sectional views of the vicinity of the label pad 31. FIG. 15A and FIG. 15B are plan views of the vicinity of the label pad 31 corresponding to FIG. 8A, and FIG. 8B is a cross-sectional view taken along line b-b in FIG. 15 (a).

In the example shown in FIG. 11, the first absorbent body 121 is disposed to be separated from the inner bottom surface 12A of the case main body 12, and the second absorbent body 122 is disposed directly below the first absorbent body 121. As indicated by an arrow 88 in FIG. 11, the sample solution 72 absorbed by the first absorbent body 121 leaks from a lower end 121a of the first absorbent body 121 by a self-weight and is absorbed by the second absorbent body 122 disposed directly below the first absorbent body 121. According to the present configuration, the sample solution 72 can be suppressed from spreading on the inner bottom surface 12A of the case main body 12, and the sample solution 72 can be efficiently absorbed by the second absorbent body 122.

In the example shown in FIG. 12, a first absorbent body 131 is disposed such that a lower end 131a thereof is in contact with the inner bottom surface 12A of the case main body 12. In addition, as the second absorbent body 132, two absorbent bodies 132A and 132B (hereinafter, referred to as a second absorbent body 132A and a second absorbent body 132B) are disposed on the upstream side and the downstream side of the first absorbent body 131. The sample solution 72 absorbed by the first absorbent body 131 moves vertically downward in the first absorbent body 131 by a capillary force and/or a self-weight as indicated by an arrow 86 in FIG. 12B. The sample solution 72 that has leaked from the lower end 131a of the first absorbent body 131 and has spread on the inner bottom surface 12A of the case main body 12 may spread on both the upstream side and the downstream side.

Since the second absorbent bodies 132A and 132B are disposed on the upstream side and the downstream side, the sample solution 72 that has leaked to the inner bottom surface 12A can be absorbed by the second absorbent bodies 132A and 132B on the upstream side and the downstream side as indicated by an arrow 87A and an arrow 87B in FIG. 12B.

As described above, in a case where the second absorbent body 132 is composed of a plurality of absorbent bodies 132A and 132B disposed around the first absorbent body 131, the excess sample solution 72 that has leaked from the first absorbent body 131 can be quickly and accurately absorbed.

It is noted that, in the above-described example, the second absorbent body 132 is composed of two absorbent bodies 132A and 132B, but the second absorbent body 132 may be composed of three or more absorbent bodies. In addition, the disposition of the plurality of absorbent bodies 132A and 132B are not limited to the upstream side and the downstream side, and the plurality of absorbent bodies 132A and 132B may be disposed in parallel to the first absorbent body 131 in the X direction of the first absorbent body 131.

In the example shown in FIG. 13, the first absorbent body 131 is the same as that shown in FIG. 12. The second absorbent body 142 has one side of a length Ld equal to or longer than a length Lc of the first absorbent body 131 along the longitudinal direction (Y direction in FIG. 13) of the assay strip 14. The second absorbent body 142 is disposed such that a length Ld direction, that is, a side having the length Ld is along the longitudinal direction of the assay strip 14, and in an orientation in which a surface having the side of the length Ld faces the first absorbent body 131.

In a case where the second absorbent body 142 has a length Ld equal to or longer than the length Lc of the first absorbent body 131 and is disposed to face the first absorbent body 131, the excess sample solution 72 that has leaked from the first absorbent body 131 can be quickly absorbed.

In the example shown in FIG. 14, the first absorbent body 131 is the same as that shown in FIGS. 12 and 13.

The second absorbent body 152 has a shape surrounding the first absorbent body 131. In the present example, the second absorbent body 152 has a U-shape. It is noted that the shape surrounding the periphery refers to a shape in which the second absorbent body 152 has a portion facing at least two surfaces of the first absorbent body 131. Therefore, the second absorbent body 152 is not limited to the U-shape, and may have an L-shape or a shape surrounding the first absorbent body 131 in a ring shape.

As described above, in a case where the second absorbent body 152 has a shape surrounding the first absorbent body 131, the excess sample solution 72 that has leaked from the first absorbent body 131 can be quickly and accurately absorbed.

In the example shown in FIG. 15, a first absorbent body 161 consists of two absorbent bodies 161A and 161B (hereinafter, referred to as a first absorbent body 161A and a first absorbent body 161B), and a second absorbent body 162 consists of two absorbent bodies 162A and 162B (hereinafter, referred to as a second absorbent body 162A and a second absorbent body 162B). As shown in FIG. 15, the first absorbent body 161A and the first absorbent body 161B are disposed on both sides of the assay strip 14, with the assay strip interposed therebetween, and the second absorbent body 162A and the second absorbent body 162B are disposed on both sides of the assay strip 14, with the assay strip interposed therebetween. The first absorbent body 161A and the first absorbent body 161B are disposed in the same shape and orientation as the first absorbent body 131 shown in FIG. 12. The second absorbent body 162A and the second absorbent body 162B are disposed in the same shape and orientation as the U-shaped second absorbent body 152 shown in FIG. 14.

As described above, in a case where the first absorbent bodies 161A and 161B and the second absorbent bodies 162A and 162B are disposed to sandwich the assay strip 14, the excess sample solution 72 can be absorbed from both sides of the label pad 31 in the width direction. In addition, since the sample solution 72 absorbed by the first absorbent body 161A and the first absorbent body 161B is returned to the label pad 31 from both sides in the width direction, the sample solution 72 can pass through both sides of the label pad 31 evenly as compared with a case where the sample solution 72 is disposed on only one side of the label pad 31, so that the reaction between the test substance 73 and the labeling substance 70 can be promoted.

The contents described and shown above are detailed descriptions of portions according to the technology of the present disclosure and are merely examples of the technology of the present disclosure. For example, the above description of the configurations, functions, operations, and effects is the description of examples of the configurations, functions, operations, and effects of portions related to the technology of the present disclosure. Therefore, unnecessary portions may be deleted or new elements may be added or replaced in the above descriptions and illustrations without departing from the gist of the technology of the present disclosure. In addition, to avoid complication and facilitate understanding of portions according to the technology of the present disclosure, description related to common technical knowledge or the like that does not need to be particularly described for enabling implementation of the technology of the present disclosure is omitted in the contents described and shown above.

The disclosure of Japanese Patent Application No. 2023-119417 filed on Jul. 21, 2023 is incorporated herein by reference in its entirety.

All documents, patent applications, and technical standards disclosed in the present specification are incorporated in the present specification by reference to the same extent as those in a case where each of the documents, patent applications, and technical standards are specifically and individually indicated to be incorporated by reference.

In regard to the above-described embodiment, following appendixes are further disclosed.

### Appendix 1

An immunochromatographic assay device comprising:
an assay strip on which a sample solution is developed, the strip including an assay region configured to capture a test substance contained in the sample solution, and a label pad on an upstream side of the assay region, the label pad containing a labeling substance that labels the test substance;
a case that accommodates the assay strip and includes a cover member including a dropping port configured to add the sample solution dropwise onto the label pad of the assay strip, and a case main body configured to accommodate the assay strip and including a recessed portion and a support table on an inner bottom surface of the recessed portion, the support table being configured to support the assay strip above the inner bottom surface;
a first absorbent body that is disposed, on a side of the assay strip in a width direction, in contact with at least a side surface of the label pad and in an orientation extending toward an inner bottom surface side of the case main body, the first absorbent body being configured to absorb the sample solution by a capillary force; and
a second absorbent body that is disposed, on the inner bottom surface of the case main body, at a position separated from the assay strip and the first absorbent body, the second absorbent body being configured to absorb the sample solution that has leaked from the first absorbent body.

### Appendix 2

The immunochromatographic assay device according to appendix 1,
wherein a liquid-holding capacity of the first absorbent body is equal to or less than a specified amount to be added dropwise onto the assay strip.

### Appendix 3

The immunochromatographic assay device according to appendix 1 or 2,
wherein a total liquid-holding capacity of the label pad and the first absorbent body is 1 to 2 times a specified amount to be added dropwise onto the assay strip.

### Appendix 4

The immunochromatographic assay device according to any one of appendixes 1 to 3,
wherein the first absorbent body consists of the same material as a material of the label pad.

### Appendix 5

The immunochromatographic assay device according to any one of appendixes 1 to 4,
wherein a contact portion between the first absorbent body and the assay strip is only the label pad.

### Appendix 6

The immunochromatographic assay device according to any one of appendixes 1 to 5,
wherein the first absorbent body includes the labeling substance.

### Appendix 7

The immunochromatographic assay device according to any one of appendixes 1 to 6,
wherein the second absorbent body is composed of a plurality of absorbent bodies disposed around the first absorbent body.

### Appendix 8

The immunochromatographic assay device according to any one of appendixes 1 to 7,
wherein the second absorbent body has a length equal to or longer than a length of the first absorbent body along a longitudinal direction of the assay strip, and is disposed with a lengthwise direction of the second absorbent body along the longitudinal direction and facing to the first absorbent body.

### Appendix 9

The immunochromatographic assay device according to any one of appendixes 1 to 8,
wherein the first absorbent body is in contact with the inner bottom surface of the case main body.

### Appendix 10

The immunochromatographic assay device according to any one of appendixes 1 to 8,
wherein the first absorbent body is disposed to be separated from the inner bottom surface of the case main body, and
the second absorbent body is disposed directly below the first absorbent body.

### Appendix 11

The immunochromatographic assay device according to any one of appendixes 1 to 9,
wherein the second absorbent body has a shape surrounding the first absorbent body.

### Appendix 12

The immunochromatographic assay device according to any one of appendixes 1 to 11,
wherein the second absorbent body is disposed at a position at a distance of 1 mm or less from the first absorbent body.

### Appendix 13

The immunochromatographic assay device according to any one of appendixes 1 to 12,
wherein the first absorbent body and the second absorbent body are disposed on both sides of the assay strip, with the assay strip interposed therebetween.

What is claimed is:

1. An immunochromatographic assay device comprising:
an assay strip on which a sample solution is developed, the strip including an assay region configured to capture a test substance contained in the sample solution, and a label pad on an upstream side of the assay region, the label pad containing a labeling substance that labels the test substance;
a case that accommodates the assay strip and includes a cover member including a dropping port configured to add the sample solution dropwise onto the label pad of the assay strip, and a case main body configured to accommodate the assay strip and including a recessed portion and a support table on an inner bottom surface of the recessed portion, the support table being configured to support the assay strip above the inner bottom surface;
a first absorbent body that is disposed, on a side of the assay strip in a width direction, in contact with at least a side surface of the label pad and in an orientation extending toward an inner bottom surface side of the case main body, the first absorbent body being configured to absorb the sample solution by a capillary force; and
a second absorbent body that is disposed, on the inner bottom surface of the case main body, at a position separated from the assay strip and the first absorbent body, the second absorbent body being configured to absorb the sample solution that has leaked from the first absorbent body.

2. The immunochromatographic assay device according to claim 1,
wherein a liquid-holding capacity of the first absorbent body is equal to or less than a specified amount to be added dropwise onto the assay strip.

3. The immunochromatographic assay device according to claim 1,
wherein a total liquid-holding capacity of the label pad and the first absorbent body is 1 to 2 times a specified amount to be added dropwise onto the assay strip.

4. The immunochromatographic assay device according to claim 1,
wherein the first absorbent body consists of the same material as a material of the label pad

5. The immunochromatographic assay device according to claim 1,
wherein a contact portion between the first absorbent body and the assay strip is only the label pad.

6. The immunochromatographic assay device according to claim 1,
wherein the first absorbent body includes the labeling substance.

7. The immunochromatographic assay device according to claim 1,
wherein the second absorbent body is composed of a plurality of absorbent bodies disposed around the first absorbent body.

8. The immunochromatographic assay device according to claim 1,
wherein the second absorbent body has a length equal to or longer than a length of the first absorbent body along a longitudinal direction of the assay strip, and is disposed with a lengthwise direction of the second absorbent body along the longitudinal direction and facing to the first absorbent body.

9. The immunochromatographic assay device according to claim 1,
wherein the first absorbent body is in contact with the inner bottom surface of the case main body.

10. The immunochromatographic assay device according to claim 1,
wherein the first absorbent body is disposed to be separated from the inner bottom surface of the case main body, and
the second absorbent body is disposed directly below the first absorbent body.

11. The immunochromatographic assay device according to claim 1,
wherein the second absorbent body has a shape surrounding the first absorbent body.

12. The immunochromatographic assay device according to claim 1,
wherein the second absorbent body is disposed at a position at a distance of 1 mm or less from the first absorbent body.

13. The immunochromatographic assay device according to claim 1,
wherein the first absorbent body and the second absorbent body are disposed on both sides of the assay strip, with the assay strip interposed therebetween.
